(19) United States
(12) Patent Application Publication
(10) Pub. No.: US 2014/0228641 A1
(43) Pub. Date: Aug. 14, 2014

(54) DIAGNOSTIC CAPSULES, DELIVERY / RETRIEVAL SYSTEMS, KITS AND METHODS

(71) Applicant: Mayo Foundation for Medical Education and Research, Rochester, MN (US)

(72) Inventor: Matthew T. Gettman, Rochester, MN (US)

(21) Appl. No.: 14/257,645

(22) Filed: Apr. 21, 2014

Related U.S. Application Data
(63) Continuation of application No. 13/129,480, filed on Aug. 1, 2011.
(60) Provisional application No. 61/115,388, filed on Nov. 17, 2008.

(51) Int. Cl. A61B 1/04; A61B 5/07

(52) U.S. Cl. CPC A61B 1/041; A61B 5/07
USPC 600/115; 600/114

(57) ABSTRACT

Diagnostic capsules, a variety of diagnostic capsule delivery systems for delivering a diagnostic capsule into an animal or human body cavity (e.g., into an animal or human bladder through a urethra), a variety of diagnostic capsule retrieval systems, a variety of kits that include diagnostic capsule delivery and retrieval systems and diagnostic capsules, and methods of using the same are described herein.

### RELATED APPLICATION

The present application claims the benefit under 35 U.S.C. §119(e) of U.S. Provisional Patent Application No. 61/115,388, titled DIAGNOSTIC CAPSULES AND METHODS OF USING SAME, filed on Nov. 17, 2008, which is hereby incorporated by reference.

The present invention relates to a medical device (e.g., a diagnostic capsule) for use in internal medical diagnostics and methods of using same.

Medical devices in the form of capsules having, for example, sensors and imaging devices, have been used in gastroenterological procedures. Capsules including, for example, endoscopic and other equipment have been described in U.S. Pat. No. 5,604,531 (Iddan et al.), U.S. Pat. No. 6,240,312 (Alfano et al.), and U.S. Pat. No. 7,039,453 (Mullick et al.), as well as in U.S. Patent Application Publication Nos. 2007/0083083 (Mori et al.), 2004/0199054 (Wakefield), 2005/0165272 (Okada et al.), 2008/0249360 (Li et al.), and 2005/0148842 (Wang et al.), 2005/0192478 (Williams et al.). U.S. Pat. No. 6,293,923 (Yachia et al.) describes an expandable balloon for treating urinary incontinence. However, improved medical devices (e.g., capsules), systems, and methods are sought.

### SUMMARY

Diagnostic capsules, a variety of diagnostic capsule delivery systems for delivering a diagnostic capsule into an animal or human body cavity (e.g., into an animal or human bladder through a urethra), a variety of diagnostic capsule retrieval systems, a variety of kits that include diagnostic capsule delivery and retrieval systems and diagnostic capsules, and methods of using the same are described herein.

Although the diagnostic capsules described herein may be delivered into a body lumen including a urinary tract and bladder, the diagnostic capsules may be utilized in a wide variety of body lumens (e.g., nasal, arterial, oral, esophageal, intestinal, etc.). Further, the diagnostic capsules of the present disclosure may include a wide variety of diagnostic devices.

Treatment of, for example, a urinary tract or a bladder may be challenging due to, for example, limitations of conventional equipment. For example, some equipment may be bulky and may cause patient discomfort relating to passage through a patient's urethra. Some equipment, for example, does not allow the patient to evacuate the bladder during such examinations.

In one aspect, embodiments of a diagnostic capsule as described herein include a housing wherein the housing comprises a restrained linear configuration at least when located within a lumen of a delivery device and an unrestrained curvilinear configuration when located outside of the lumen; and an imaging device located within the housing.

In some embodiments, the diagnostic capsules described herein may include an eyelet on an exterior of the housing, wherein the eyelet comprises an opening for attachment to a retrieval article adapted to extend through a body lumen to retrieve the diagnostic capsule.

In some embodiments, the diagnostic capsules described herein may include a length of suture material threaded through the eyelet.

In some embodiments, the diagnostic capsules described herein may include a housing that, in the unrestrained curvilinear configuration, comprises a U-shaped configuration. In some embodiments, the diagnostic capsule further comprises two imaging devices, wherein the two imaging devices are positioned at opposite ends of the housing, and wherein the two imaging devices are oriented in the same imaging direction when the housing is in the U-shaped configuration.

In some embodiments, the diagnostic capsules described herein may include a magnetic positioning element.

In some embodiments, the diagnostic capsules described herein may include a housing that, in the unrestrained curvilinear configuration, comprises a U-shaped configuration, and wherein the diagnostic capsule further comprises two imaging devices, wherein the two imaging devices are positioned at opposite distal ends of the housing, and wherein the two imaging devices are oriented in the same imaging direction when the housing is in the U-shaped configuration, and further wherein the diagnostic capsule comprises a magnetic positioning element located between the two imaging devices. In some embodiments, the magnetic positioning element is located at an apex of the housing when the housing is in the U-shaped configuration.

In another aspect, a diagnostic capsule delivery system may be provided that includes a sheath having a proximal end, a distal end, and a first lumen extending therethrough; a diagnostic capsule comprising a housing that comprises a restrained linear configuration at least when located within the first lumen of the sheath and an unrestrained curvilinear configuration when located outside of the first lumen, wherein the diagnostic capsule comprises an imaging device located within the housing; and an elongate obturator sized to advance through the first lumen to move the diagnostic capsule through the first lumen.

In another aspect, a diagnostic capsule delivery system may be provided that includes a diagnostic capsule comprising a housing and an eyelet formed in or otherwise attached to the housing; an elongate obturator; a sheath having a proximal end, a distal end, and a first lumen extending therethrough, wherein the first lumen is sized to allow passage of the obturator and the diagnostic capsule through the first lumen; and a retrieval article attached to the eyelet of the diagnostic capsule, wherein the retrieval article comprises a length sufficient to extends through the first lumen from the proximal end to the distal end of the sheath.

In some embodiments of the diagnostic capsule delivery systems described herein, the obturator comprises a proximal end, a distal end, and a second lumen extending through the obturator between the proximal end and the distal end of the obturator.

In some embodiments of the diagnostic capsule delivery systems described herein, the obturator comprises a proximal end, a distal end, and a magnetic positioning element proximate the distal end of the obturator, and wherein the diagnostic element comprises magnetic positioning element such that the diagnostic element can be magnetically attracted to the distal end of the obturator.

In some embodiments of the diagnostic capsule delivery systems described herein, the housing of the diagnostic capsule is cradled in the distal end of the obturator.

In some embodiments of the diagnostic capsule delivery systems described herein, the distal end of the obturator comprises a concave shape and the housing of the diagnostic capsule comprises a convex portion complementary to the concave shape.

In some embodiments of the diagnostic capsule delivery systems described herein, the housing, in the unrestrained curvilinear configuration, comprises a U-shaped configuration. In some embodiments, the diagnostic capsule comprises two imaging devices, wherein the two imaging devices are positioned at opposite ends of the housing, and wherein the two imaging devices are oriented in the same imaging direction when the housing is in the U-shaped configuration.

In some embodiments of the diagnostic capsule delivery systems described herein, the diagnostic capsule further comprises a magnetic positioning element.

In some embodiments of the diagnostic capsule delivery systems described herein, the housing of the diagnostic capsule, in the unrestrained curvilinear configuration, comprises a U-shaped configuration, and the diagnostic capsule further comprises two imaging devices, wherein the two imaging devices are positioned at opposite ends of the housing, and the two imaging devices are oriented in the same imaging direction when the housing is in the U-shaped configuration, and further the diagnostic capsule comprises a magnetic positioning element. In some embodiments, the magnetic positioning element is located at an apex of the housing when the housing is in the U-shaped configuration.

In some embodiments of the diagnostic capsule delivery systems described herein, the retrieval article is threaded through the second lumen of the obturator.

In some embodiments of the diagnostic capsule delivery systems described herein, the diagnostic capsule comprises a channel formed through the housing, and wherein the system further comprises an inflatable balloon comprising a neck portion located within the channel.

In some embodiments of the diagnostic capsule delivery systems described herein, the diagnostic capsule further comprises a power source located within the housing, the power source being operably connected to the imaging device.

In some embodiments of the diagnostic capsule delivery systems described herein, the diagnostic capsule further comprises a light emitting element.

In some embodiments of the diagnostic capsule delivery systems described herein, the diagnostic capsule further comprises a specimen collection reservoir within the housing.

In some embodiments of the diagnostic capsule delivery systems described herein, the diagnostic capsule further comprises a transmitter within the housing, wherein the transmitter is operable to transmit data obtained by the imaging device to a receiver located outside of the housing. In some embodiments, the transmitter comprises a wireless transmitter.

In another aspect, some embodiments of a diagnostic capsule as described herein may include a housing; an imaging device located within the housing; and an eyelet on an exterior of the housing, wherein the eyelet comprises an opening for attachment to a retrieval article adapted to extend through a body lumen to retrieve the diagnostic capsule. In some embodiments, the diagnostic capsule may include a length of suture material threaded through the eyelet.

In another aspect, some embodiments of a diagnostic capsule delivery system as described herein may include a sheath having a proximal end, a distal end, and a first lumen extending therethrough; a balloon adapted for passing through the sheath while at least partially inflated, wherein the balloon has an inflatable end and a neck end; the diagnostic capsule as described herein further comprising a through-channel capable of receiving the neck end of the balloon, wherein the neck end extends through the through-channel of the diagnostic capsule; an elongate obturator; wherein the sheath is capable of receiving the obturator and the diagnostic capsule, wherein the sheath is optionally capable of extending from inside an animal's body to outside an animal's body, wherein inside the animal's body optionally comprises inside the animal's bladder, wherein the obturator is capable of extending from the proximal end of the sheath to the distal end of the sheath, wherein the obturator optionally comprises a second lumen for an optional passage of fluid, and wherein the passage of fluid is optionally into or out of an animal's bladder; wherein the passage of fluid is optionally into or out of the balloon; and an optional retrieval article attached to the eyelet, wherein the retrieval article is adapted to extend through a body lumen to permit retrieval of the diagnostic capsule.

The words “preferred” and “preferably” as used herein refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably. Thus, for example, an imaging device may be used to refer to one, two, three or more imaging devices.

The term “and/or” means one or all of the listed elements or a combination of any two or more of the listed elements.

The above summary is not intended to describe each embodiment or every implementation of the health care delivery monitoring systems and methods described herein. Rather, a more complete understanding of the health care delivery monitoring systems described herein will become apparent and appreciated by reference to the following Description of Illustrative Embodiments and claims in view of the accompanying figures of the drawing.

### BRIEF DESCRIPTIONS OF THE FIGURES

FIG. 1 depicts one illustrative embodiment of a diagnostic capsule including an eyelet.

FIG. 2 depicts another illustrative embodiment of a diagnostic capsule including an eyelet and a through-channel

FIG. 3A depicts another illustrative embodiment of a diagnostic capsule including an eyelet and an elongate configuration.

FIG. 3B depicts one illustrative embodiment of a diagnostic capsule including a curvilinear configuration.

FIG. 3C depicts another illustrative embodiment of a diagnostic capsule including a curvilinear configuration.

FIG. 3D depicts another illustrative embodiment of a diagnostic capsule including a restrained configuration within the lumen of a delivery device.

FIG. 3E depicts the diagnostic capsule of FIG. 3D in an unrestrained curvilinear configuration.

FIG. 4 depicts another illustrative embodiment of a diagnostic capsule.

FIGS. 5A and 5B depict an illustrative embodiment of a diagnostic capsule delivery system using a balloon with a diagnostic capsule.

FIGS. 6A and 6B depict another illustrative embodiment of a diagnostic capsule delivery system.

FIG. 7 depicts an illustrative embodiment of a diagnostic capsule retrieval system.

FIG. 8 depicts another illustrative embodiment of a diagnostic capsule retrieval system.

FIG. 9 is a block diagram of an optional undergarment that may include a plurality of magnets to assist in movement of a diagnostic capsule as described herein.

### DESCRIPTION OF ILLUSTRATIVE EMBODIMENTS

In the following description of illustrative embodiments, reference is made to the accompanying figures of the drawing which form a part hereof, and in which are shown, by way of illustration, specific embodiments in which the systems and/or methods may be practiced. It is to be understood that other embodiments may be utilized and structural changes may be made without departing from the scope of the present invention:

The diagnostic capsules, systems, kits, and methods described herein may provide various advantages, such as decreasing patient discomfort, reducing required specialist time, and improved examination data.

Such devices could be useful for remote tele-endoscopy whereby the patient could have the capsule deployed by a nurse or other health care provider (potentially even in the home) and have the images transmitted to a remote facility using any suitable data transmission system (e.g., the Internet, etc.). Such devices could also eliminate costs of acquiring, sterilizing, and maintaining traditional endoscopic equipment as well as office space and personnel required to perform such tasks. In this scenario, a physician located remotely from the patient could receive the images and other data, provide a diagnosis or other evaluation, and obtain the professional fee for this service without requiring face-to-face interaction between the doctor and the patient.

Some embodiments of the diagnostic capsules described herein include a housing, an eyelet, and a variety of optional components.

The housing may be provided in any appropriate shape, size, and configuration, for example, for insertion into and passage through a urethra into a bladder. For example, a housing with rounded edges and rounded corners may be used. A housing may also be substantially spherical, substantially elongate, etc. A spherical design may, for example, provide improved visualization in the bladder. For example, since the bladder is spherical, cameras or other imaging devices could be placed circumferentially about the housing. In one or more embodiments, a portion of the housing may be hemi-ellipsoidal. The housing may include a distal end and a proximal end. In other embodiments, the housing may also be cylindrical or elongated, thereby providing an elongated shape

A housing for a diagnostic capsule as described herein may be constructed from one or more of a wide variety of suitable materials. For example, a housing may be constructed of metallic materials, silicon-based (e.g., silicone-based) materials, polymer-based materials, ceramics, etc. One having ordinary skill in the art will recognize other suitable housing materials. In some embodiments, the housing could be coated with or contain substances to reduce infection and/or provide diagnosis or treatment of disorders within the deployed organ. The housing material could, in some embodiments, also be suitable for a drug-eluting function to deliver one or more drugs or other medical substances to a patient directly from the capsule.

In one or more embodiments, the housing may be adapted to be advanced through an elongate passage (e.g., a urinary tract) for use in, for example, a diagnostic procedure.

A diagnostic capsule as described herein may include, in some embodiments, an eyelet formed in or otherwise attached to an exterior of the housing. For example, if the eyelet is formed in the housing, the eyelet may include a passage through the housing material though which a deployment and/or retrieval article may be attached to the capsule. In one or more embodiments, the retrieval article may be a suture threaded through the eyelet. In one or more embodiments, the eyelet may include a ring, loop, hookeye, hook, etc. that is otherwise attached to the housing. For example, a ring or hook may receive a retrieval article therethrough and may be attached to the housing by any type of connector.

In one or more embodiments, a diagnostic capsule as described herein may include an optional retrieval article. For example, a suitable retrieval article may include, but is not limited to, a suture, a string, a thread, a wire, a chain, a power line, etc. The retrieval article may have any suitable diameter such that the retrieval article can be threaded through an eyelet formed in or otherwise attached to the housing.

In one or more embodiments, the retrieval article may be non-absorbable, antibacterial, coated, coiled, braided, etc. In the present disclosure, “non-absorbable” means that the retrieval article (e.g., a suture) will not be absorbed by or dissolve during normal use while in contact with a body fluid or tissue. A retrieval article that is antibacterial may have an antibacterial component embedded within or otherwise attached to the retrieval article. In one or more embodiments, a retrieval article may be coated with a polymer-based material, a metal, an alloy, or some other material. For example, a coating on a retrieval article may provide characteristics to the retrieval article including, but not limited to, lubricity, rigidity, tensile strength, chemical resistance, and/or other characteristics. In one or more embodiments, more than one (e.g., two or more, three or more, etc.) retrieval article may be used and may be coiled (e.g., a double helix configuration) or may be braided. A retrieval article must have any suitable tensile strength such that pulling the retrieval article results in pulling the diagnostic capsule, for example, through a urinary channel.

A retrieval article may be constructed from one or more of a wide variety of suitable materials. Some potentially suitable retrieval article materials may include, e.g., but are not limited to, natural or synthetic materials, polymer-based materials, metals, and alloys. One having ordinary skill in the art will recognize other suitable retrieval article materials.

The incorporation of an eyelet (e.g., hookeye, hook, loop, or ring) on an exterior of the housing of a diagnostic capsule for use in, for example, a bladder, wherein a retrieval article is secured to the eyelet may have advantages in that retrieval of the diagnostic capsule may be accomplished with reduced discomfort to the patient and without the need for a sheath or other delivery device to remain in place during, for example, collection of data by the diagnostic capsule.

In one or more embodiments, a diagnostic capsule as described herein may include a magnetic positioning element within or connected to the housing. As used herein, a “magnetic positioning element” includes material that is permanently magnetic and/or material that is susceptible to magnetic attraction. A magnetic positioning element may emit a magnetic field and may be capable of attracting other magnetic materials (e.g., metals or other magnets). In one or more embodiments, a magnetic positioning element may include materials that are permanent magnets and materials that may be magnetized. Magnetic materials include, but are not limited to, iron, nickel, boron, cobalt, rare earth metals and combinations thereof. One having ordinary skill in the art would recognize other suitable magnetic materials that may be include in a magnetic positioning element within or connected to the housing. Depending on the specific use or intended movement of the diagnostic capsule, one or more magnetic positioning elements may be provided in connection with each diagnostic capsule, and the arrangement/positioning of the magnetic positioning elements can be selected to provide the desired positioning performance.

Externally, a diagnostic capsule including one or more magnetic positioning elements may preferably be moved with a magnetic field generator that is placed on the skin surface of the subject. The capsule movements may also be accomplished by more sophisticated arrangements of magnetic field generators or with the use of specialized instruments that would move the magnetic positioning element as part of a standardized protocol that would be computer controlled. Undergarments equipped with magnets and required data capturing devices may also be incorporated into the invention. For example, selective switching of magnetic fields within the magnetic undergarments may facilitate movement of the capsule in a standardized fashion within the deployed organ. Such magnetic undergarments may include a plurality of magnetic field generators that may be selectively actuated (e.g., electromagnets operatively connected to a control system, etc.) to provide forces (attractive and/or repulsive) to cause the capsule to move in a selected direction.

The magnetic properties of the capsule may also be augmented for diagnostic and/or therapeutic purposes by pre-treating the tissues of the body cavity. For instance, an iron emulsion could be instilled into the body cavity as a way to augment magnet movement of the capsule. For diagnostic and potentially therapeutic implications related to, e.g., bladder cancer, antibodies to cancer cells could be tagged to the iron particles. Pretreatment with the emulsion would cause antibodies with iron particles to attach to the tumor cells. Deployment of the capsule would then be preferential to identification of these areas and also treating these areas. Other applications of the same concept could be envisioned for conditions other than bladder cancer.

In one or more embodiments, a diagnostic capsule as described herein may include one or more light emitting elements within the housing. As used herein, a “light emitting element” includes any device that can emit any wavelength of electromagnetic energy (e.g., any light in visible spectrum, ultraviolet light, infrared light, etc.) that can be used in connection with other diagnostic devices on or in the diagnostic capsule (e.g., an imaging device such as a camera, etc.). A diagnostic capsule may include more than one light emitting element wherein the light emitted by each light emitting element is of the same or different wavelength. In one or more embodiments, the one or more light emitting elements may include electronics capable of turning any of the one or more light emitting elements on or off. In one or more embodiments, an light emitting element (e.g., a light mechanism, like a strobe) may be used with or without sensitizing agents to aid in identifying tumors. In one or more embodiments, multiple light emitting elements (e.g., similar to track lighting) may be provided on a diagnostic capsule in order to illuminate an area of potential interest (e.g., for image retrieval). The light emitting elements of the capsules may also be used in connection with surgical navigation systems. For instance, capsules may be used to identify surgical areas of interest during surgical procedures. Identification of the capsules using light could be used to accomplish this goal.

In one or more embodiments, a diagnostic capsule as described herein may include one or more optional imaging devices within the housing. For example, the one or more imaging devices may include a camera disposed within the housing capable of capturing images outside the diagnostic capsule (e.g., of a bladder lining). In one or more embodiments, the one or more imaging devices may be capable of obtaining still images and/or or video images, which may be captured at any suitable number of frames per second (e.g., one or more, two or more, five or more, ten or more, or 20 or more frames per second). The one or more optional imaging devices may be adapted to capture images in a low light intensity environment. In one or more embodiments that include a plurality of imaging devices, the field of vision for each imaging device may or may not overlap and may be directed in completely opposite directions, directed at right angles, or directed at some other angle relative to another imaging device. In one or more embodiments, a housing may include a hemi-ellipsoidal lens or dome covering the imaging device portion of the diagnostic capsule, wherein the hemi-ellipsoidal shape of the housing may, for example, aid introduction of the diagnostic capsule into a urethra. In one or more embodiments, the diagnostic capsule including one or more optional imaging devices may be used as a camera for intralumenal NOTES applications.

In one or more embodiments, a diagnostic capsule as described herein may include one or more optional specimen collection reservoirs within the housing. For example, the one or more specimen collection reservoirs may include an optional one-way valve to allow only the input of fluid while the diagnostic capsule is within a body cavity having fluid therein, wherein the one-way valve may be otherwise manipulated to access the collected specimen when the diagnostic capsule is not located within a body cavity. In one or more embodiments wherein the diagnostic capsule is located within a body cavity, the one or more optional specimen collection reservoirs may be used to collect proximal body fluids (e.g., passive collection of sterile urine when the diagnostic capsule is located in, for example, a bladder) or body tissue. One having ordinary skill in the art will recognize other suitable types of specimen collection reservoirs and corresponding optional specimen collection equipment. In one or more embodiments, the specimen collection reservoirs may be any suitable size (e.g., 0 to 10 cubic centimeters, 0 to 2 cubic centimeters, etc.).

In one or more embodiments, a diagnostic capsule as described herein may include one or more optional sensors within the housing. For example, the one or more sensors may include one or more of the following optional sensors: a pressure manometer, an impedance sensor (e.g., may be useful for identifying tumors), a biomarker detection sensor, and a temperature sensor. In one or more embodiments wherein the diagnostic capsule is located within a body cavity (e.g., a bladder), the one or more optional sensors may be used to, for example, measure and collect data regarding pressure, temperature, light, and other physical or chemical characteristics of the environment or properties of the luminal wall. One having ordinary skill in the art will recognize other suitable types of sensors and corresponding optional sensor equipment that may be useful in a diagnostic capsule of the present disclosure. In one or more embodiments, a diagnostic capsule may include none, one, or more than one of any of the optional sensors. For example, a diagnostic capsule may include several manometer sensors to measure pressure, which may be useful in ambulatory urodynamics testing using, e.g., wireless information technologies and interpretation of the recording by the health care provided that would be potentially remote from the patient. In one or more embodiments, a sensor may be used to detect proximal biomarkers. In one or more embodiments, the one or more optional sensors may be located within the housing in any suitable arrangement.

In one or more embodiments, a diagnostic capsule as described herein may include one or more optional diagnostic devices within the housing. For example, the one or more diagnostic devices may include one or more of the following diagnostic devices: an ultrasound device, devices to facilitate measurement of glucose metabolism, cancer recognition equipment, and optical biopsy equipment. One having ordinary skill in the art will recognize other suitable types of diagnostic devices that may be useful in a diagnostic capsule of the present disclosure. In one or more embodiments, a diagnostic capsule may include none, one, or more than one of any of the optional diagnostic devices. In one or more embodiments, the one or more optional diagnostic devices may be located within the housing in any suitable arrangement.

In one or more embodiments, a diagnostic capsule as described herein may include one or more optional transmitters within the housing. For example, one or more transmitters may be used to transmit data (e.g., pressure, temperature, images, etc.) to an optional receiver for data collection and/or real-time display. In one or more embodiments, the transmitter is capable of transmitting data wirelessly via, for example, radio frequency transmission to a receiver. In one or more embodiments, the data may be transmitted from inside a body cavity (e.g., a bladder) to an optional receiver external to the body. In one or more embodiments, the transmitter is capable of transmitting data through an optional wire to a receiver connected to the wire or otherwise capable of receiving the transmission. In one or more embodiments, the transmitter may transmit data to an optional memory device within the housing. One having ordinary skill in the art will recognize other suitable configurations of one or more optional transmitters with one or more optional receivers, that will allow receipt, collection, storage, or display of data collected and transmitted.

In one or more embodiments, a diagnostic capsule as described herein may include one or more optional motors within the housing. For example, one or more motors may be used for transport purposes. In one or more embodiments, a diagnostic capsule may be a mobile diagnostic capsule that includes a motor for transportation. In one or more embodiments, a diagnostic capsule may include one or more optional transport mechanisms within the housing or connected to the housing. In one or more embodiments, the one or more optional motors may be operatively coupled with one or more transport mechanisms within the housing. For example, a transport mechanism may include, but is not limited to, a tail fin, a propeller, or other transport structures. In one or more embodiments, an optional motor and optional drive mechanism may be used to provide locomotion and may control movement of the motorized diagnostic capsule.

In one or more embodiments, a diagnostic capsule as described herein may include one or more power sources. For example, one or more power sources may be useful to power, for example, the one or more optional motors, lights, diagnostic devices, sensors, transport mechanisms, imaging devices, transmitters and other components of the diagnostic capsule. In one or more embodiments, the power source may be located within the housing in the form of, for example, one or more batteries and/or magnets. In one or more embodiments, the power source may be external to the diagnostic capsule and may provide power to the diagnostic capsule through a wire or wirelessly.

In one or more embodiments, the systems and/or kits as described herein that include a diagnostic capsule may include an optional external magnetic field generator. An external magnetic field generator may be used to, for example, manipulate a diagnostic capsule having an optional magnetic positioning element in the housing. Such manipulation may allow moving and positioning the diagnostic capsule from outside the body when the diagnostic capsule is inside the body (e.g., inside a bladder, such as a full bladder). The optional external magnetic field generator may include any suitable magnetic material capable of providing a magnetic field wherein an internal diagnostic capsule could be manipulated.

In one or more embodiments, a diagnostic capsule as described herein may further optionally include a through-channel that may extend from a distal end of the housing to a proximal end of the housing. In one or more embodiments, other optional open channels may also pass through at least a portion of the housing. In one or more embodiments, a diagnostic capsule having a through-channel may allow deployment of the diagnostic capsule over a guidewire that extends to the location where the diagnostic capsule is to be deployed.

In one or more embodiments, a diagnostic capsule as described herein may include a housing that optionally has a restrained linear configuration at least when located within a straight lumen and an unrestrained curvilinear configuration when located outside of the lumen. For example, a housing may optionally have a restrained first configuration in which the housing may be oblong in shape and substantially linear and an unrestrained second configuration in which the housing shape is substantially curvilinear. In one or more embodiments in which the housing has a restrained configuration and an unrestrained configuration, the housing may include one or more shape memory materials (e.g., Nitinol, shape memory polymers, etc.). In one or more embodiments, the unrestrained curvilinear configuration may include a planar or non-planar coil or a different configuration which may be planar or non-planar. In one or more embodiments, the housing may have a restrained linear configuration when the diagnostic capsule is located within a straight lumen and the housing may have an unrestrained curvilinear configuration when the diagnostic capsule is located outside of the lumen.

In one or more embodiments, a diagnostic capsule having a housing in a restrained linear configuration may allow the diagnostic capsule, which may be elongate and which may have a complex three-dimensional configuration when deployed, to pass through a delivery sheath or body lumen that may be straight, while being capable of changing to the unrestrained curvilinear configuration when deployed.

In one or more embodiments, a diagnostic capsule having a housing in an unrestrained curvilinear configuration may be located within a bladder and may prevent increased pressure on the trigone, may keep the bladder lumen open, and may prevent the diagnostic capsule (e.g., a diagnostic capsule having an optional imaging device) from falling into the dependent portion of the bladder. The unrestrained curvilinear configuration may also, for example, allow for additional working space around an optional imaging device, such as a camera.

Another aspect of the present disclosure includes a diagnostic capsule that includes a housing wherein the housing includes a restrained linear configuration at least when located within a straight lumen and an unrestrained curvilinear configuration when located outside of the lumen, and wherein the diagnostic capsule may further include one or more of the optional components of the present disclosure. In one or more embodiments, a diagnostic capsule having a restrained linear configuration and an unrestrained curvilinear configuration may optionally include an eyelet capable of attachment to a retrieval article. As the capsule would be removed, pressure on the device as it is pulled into the lumen (e.g., the body lumen and/or a retrieval device lumen) would cause the capsule return to the restrained linear configuration.

Another aspect of the present disclosure is a diagnostic capsule delivery system that includes a diagnostic capsule as described herein, an elongate obturator, and a sheath. For example, the diagnostic capsule delivery system may be useful in delivering a diagnostic capsule as described herein having a housing, an optional eyelet, and optionally including a restrained linear configuration at least when located within a straight lumen and an unrestrained curvilinear configuration when located outside of the lumen, and wherein the diagnostic capsule may further include one or more of the optional components of the present disclosure.

In one or more embodiments, the diagnostic capsule delivery system includes a sheath. A sheath may have a proximal end, a distal end, and a lumen extending from the proximal end to the distal end. In one or more embodiments, the lumen in the sheath may be adapted to receive and pass therethrough the diagnostic capsule of the present disclosure and at least a portion of the elongate obturator. In one or more embodiments, the sheath may extend from inside an animal's body to outside an animal's body. For example, the sheath may be disposed within a urinary tract, for example, from the bladder through the urethra and extending outside the body.

In one or more embodiments, a diagnostic capsule delivery system as described herein may include an obturator. In the present disclosure, an obturator may be elongate and may be adapted for extending through the lumen of the sheath from the proximal end of the sheath to the distal end of the sheath. In one or more embodiments, an obturator may include a distal end having a concave portion in which, for example, a diagnostic capsule of the present disclosure may be situated. An obturator may further include an optional lumen passing along at least a portion of a longitudinal axis and extending from the distal end of the obturator toward the proximal end of the obturator. The optional lumen in the obturator may allow an optional passage of fluid therethrough (e.g., into or out of a bladder). In one or more embodiments, the passage of fluid may include, but is not limited to, the passage of saline, urine, etc.

In one or more embodiments, a diagnostic capsule delivery system as described herein may include an optional retrieval article as is described herein. In one or more embodiments, a diagnostic capsule delivery system includes a retrieval article engaging the housing via an optional eyelet, an optional through-channel, an optional loop or hook, or by some other engagement with the housing (e.g., a net around the housing). The optional retrieval article may also incorporate magnets that would permit attraction of the capsule and facilitate removal. An optional retrieval article may, for example, extend through the sheath to a location outside the body.

A diagnostic capsule may be delivered to, for example, a bladder by providing a sheath within the urinary tract from the bladder to a location outside the body. A diagnostic capsule may be placed in the proximal end of the sheath and may be advanced through the sheath by, for example, advancing an obturator against the diagnostic capsule and through the sheath. In one or more embodiments, the diagnostic capsule may be held against the distal end of the obturator by, for example, applying a pulling force on an optional retrieval article which is secured to the diagnostic capsule (e.g., to an eyelet). For example, the combination of a pulling force on the retrieval article and a pushing force on the obturator may secure the diagnostic capsule to the distal end of the obturator. In one or more embodiments, the distal end of the obturator may be shaped to cradle the diagnostic capsule (e.g., the obturator may include a concave distal end to receive, for example, a convex portion of the diagnostic capsule). In this manner, the diagnostic capsule may be advanced through the sheath and into a bladder. In one or more embodiments, the obturator may then be removed from the sheath, leaving the diagnostic capsule in the bladder and the optional retrieval article extending from the diagnostic capsule in the bladder through the urinary tract to a location outside the body. In one or more embodiments, the sheath may also be removed.

In one or more embodiments, a diagnostic capsule delivery system as described herein may include a sheath that optionally further includes a Y-shaped connector. The Y-shaped connector has a first arm, a second arm, and a connecting arm. For example, the first arm of the Y-shaped connector may include an irrigation port that may, for example, allow passage of fluids. The second arm of the Y-shaped connector may include an obturator port that may, for example, allow the passage of an obturator. In one or more embodiments, the Y-shaped connector may be integral with the sheath. In other embodiments, the Y-shaped connector may be a separate component operatively connected to the proximal end of the sheath.

Another aspect of the present disclosure is another diagnostic capsule delivery system that includes a sheath, a balloon, a diagnostic capsule as described herein, and further including a through-channel, and an elongate obturator.

In one or more embodiments, a diagnostic capsule delivery system may include a sheath as described herein. In one or more embodiments, the sheath may extend from a bladder (e.g., distal end of the sheath) to a location outside the body (e.g., proximal end of the sheath) and may further include an optional Y-shaped connector having one arm for, for example, an irrigation port and a second arm for, for example, an obturator port.

In one or more embodiments, a diagnostic capsule delivery system may include a balloon that includes an inflatable end and a neck end. For example, the neck end may receive a fluid (e.g., a gas or liquid), wherein such receipt of fluid may cause the inflatable end of the balloon to inflate. In one or more embodiments, the balloon is adapted for passing through the sheath while the inflatable end of the balloon is at least partially inflated.

In one or more embodiments, a diagnostic capsule delivery system may include an obturator as described herein, wherein the obturator may optionally include a lumen for the passage of fluid and wherein the passage of fluid may include, but is not limited to, passage of fluid into or out of the balloon or into or out of the bladder. In one or more embodiments, the elongate obturator may be capable of extending from the proximal end of the sheath to the distal end of the sheath.

In one or more embodiments, a diagnostic capsule delivery system may include a diagnostic capsule as described herein, wherein the diagnostic capsule further includes a through-channel. For example, the through-channel of the diagnostic capsule may extend from the proximal end of the diagnostic capsule to the distal end of the diagnostic capsule. Further, the through-channel may, for example, be adapted to receive the neck of a balloon. In one or more embodiments, the neck end of the balloon extends through the through-channel of the diagnostic capsule.

In the present disclosure, various kits are provided. A kit may include one or more of the following components: a diagnostic capsule delivery system, a diagnostic capsule retrieval system, the diagnostic capsule, and a magnetic field generator (e.g., an external magnet, magnetic undergarments), etc. In one or more embodiments wherein the diagnostic capsule is small enough to exit the body lumen in which it has been deployed by natural processes, a kit may be provided that includes one or more of the following components: a diagnostic capsule delivery system, a diagnostic capsule, and a magnet.

Another aspect of the present disclosure is a removal mechanism. A removal mechanism may include a diagnostic capsule as described herein having a retrieval article attached thereto. Removal may be accomplished by applying a traction force to the retrieval article and removing the diagnostic capsule. One potential removal mechanism to remove the diagnostic capsule from a bladder is by evacuation of the bladder, provided that the diagnostic capsule is small enough to pass through the urinary tract.

Another removal mechanism may include a sheath, an obturator having a magnetic distal end, and a diagnostic capsule having a component that may be attracted to the magnetic distal end of the obturator. In this removal mechanism, a sheath may be placed in a urinary tract. The obturator having the magnetic distal end may be advanced through the sheath until the distal end enters the bladder. The obturator may then be guided toward the diagnostic capsule until the diagnostic capsule engages the magnetic distal end of the obturator. Use of ultrasound may improve the guiding of the obturator to a location proximal to the diagnostic capsule. Upon engagement with the diagnostic capsule, the obturator may be withdrawn from the sheath, having the diagnostic capsule attached to the distal end of the obturator.

Another removal mechanism includes an obturator having a magnetic distal end, having a balloon extending therethrough, and having an inflatable portion of the balloon inflated proximal to the distal end of the obturator. While the balloon is inflated, the obturator may pass through the urinary tract (e.g., through a sheath in the urinary tract). When the distal end of the obturator enters the bladder, the balloon may be deflated. With the balloon in a deflated state, the magnetic distal end of the obturator may engage the diagnostic capsule. In one or more embodiments, the balloon may be withdrawn through a lumen in the obturator prior to engaging the diagnostic capsule with the magnetic distal end of the obturator.

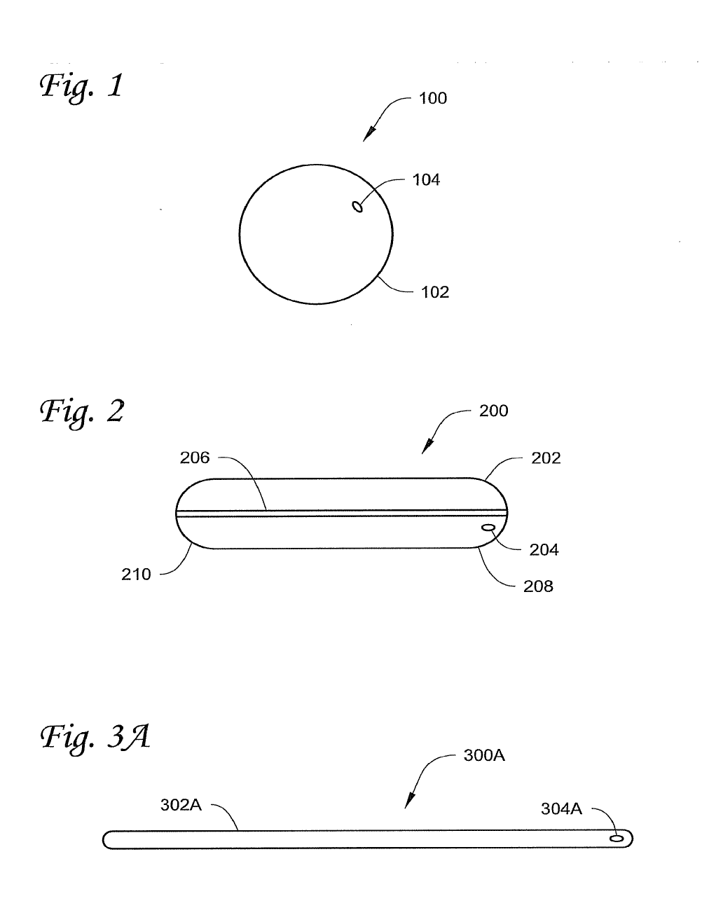

FIG. 1 shows one embodiment of a diagnostic capsule 100 according to the present disclosure. The diagnostic capsule 100 includes a spherically-shaped housing 102 and an eyelet 104.

FIG. 2 shows another diagnostic capsule 200 according to the present disclosure. The diagnostic capsule 200 includes an oblong-shaped housing 202, an eyelet 204, and a through-channel 206 extending from a proximal end 208 of the diagnostic capsule 200 to the distal end 210 of the diagnostic capsule 200. The through-channel 206 may be useful if the diagnostic capsule 200 is to be delivered over a guidewire.

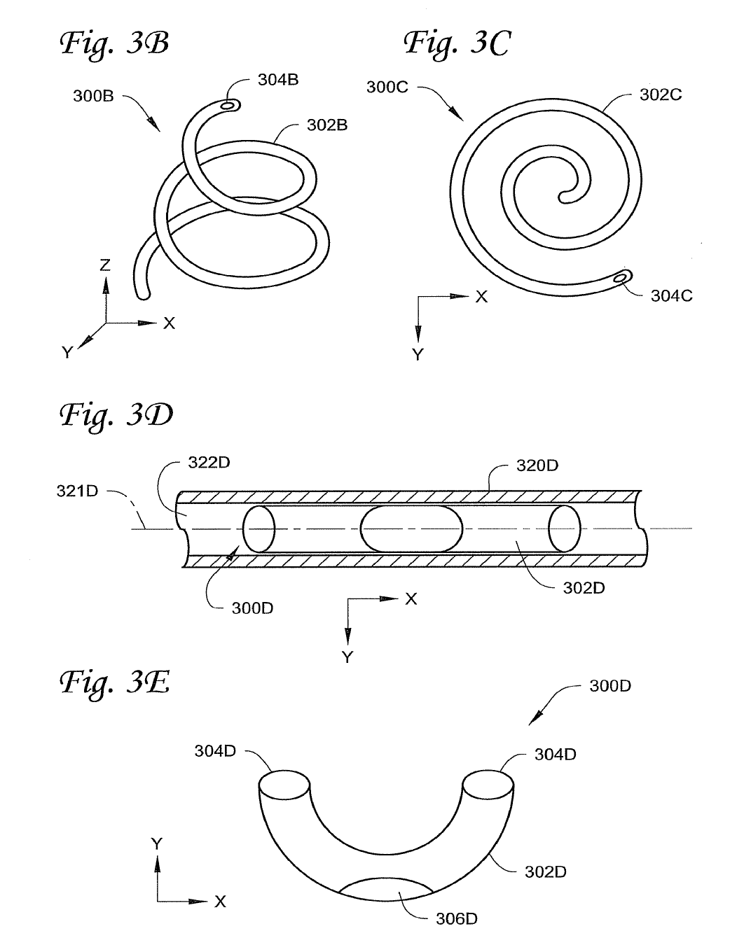

FIG. 3A shows another diagnostic capsule 300A according to the present disclosure. The diagnostic capsule 300A includes a housing 302A and an eyelet 304A. The housing 302A has a restrained linear configuration when located within a delivery lumen or sheath that is substantially straight to, e.g., assist with delivery of the diagnostic capsule 300A through a sheath, or other device as described herein.

FIGS. 3B and 3C each show an illustrative embodiment of a the capsule 300A when not restrained in a linear configuration as depicted in FIG. 3A (i.e., when in unrestrained configuration). In both FIGS. 3B and 3C, the diagnostic capsule has an unrestrained curvilinear configuration. FIG. 3B shows an embodiment of a diagnostic capsule 300B with a housing 302B and an eyelet 304B. The capsule 300B has an unrestrained curvilinear configuration in the form of a non-planar coil that extends not only in the x- and y-dimensions, but also in the z-dimension.

The unrestrained diagnostic capsule 300C depicted in FIG. 3C shows a housing 302C that takes on the shape of a planar coil that extends generally in the x- and y-dimensions and includes an eyelet 304C.

Still another embodiment of a diagnostic capsule 300D is depicted in FIGS. 3D and 3E. The diagnostic capsule 300D is depicted in a restrained configuration in FIG. 3D with the housing 302D taking on a generally linear shape along axis 321D that extends through lumen 322D of sheath 320D. After removal from the lumen 322D of sheath 320D, the diagnostic capsule 300D takes a curvilinear shape in the form of “U” or “C” as depicted in FIG. 3E. The diagnostic capsule 300D may include a pair of imaging devices (e.g., cameras, etc.) 304D on opposite ends of the housing 302D. Placement of the imaging devices 304D in a spaced-apart arrangement may be useful to provide binocular vision to enhance three-dimensional imaging of the surfaces, tissues, etc. imaged using the diagnostic capsule 300D.

Another optional feature depicted in connection with diagnostic capsule 300D is the use of one or more magnetic positioning elements 306D that can be used as described herein to manipulate the diagnostic capsule 300D. Placement of the magnetic positioning element 306D at the apex of the housing 302D may assist in pivoting or other manipulation of the diagnostic capsule 300D using magnetic field generators as discussed herein. Although only one magnetic positioning element is depicted, the diagnostic capsules as described herein may include two or more magnetic positioning elements if the additional magnetic positioning elements are useful in manipulating the diagnostic capsule.

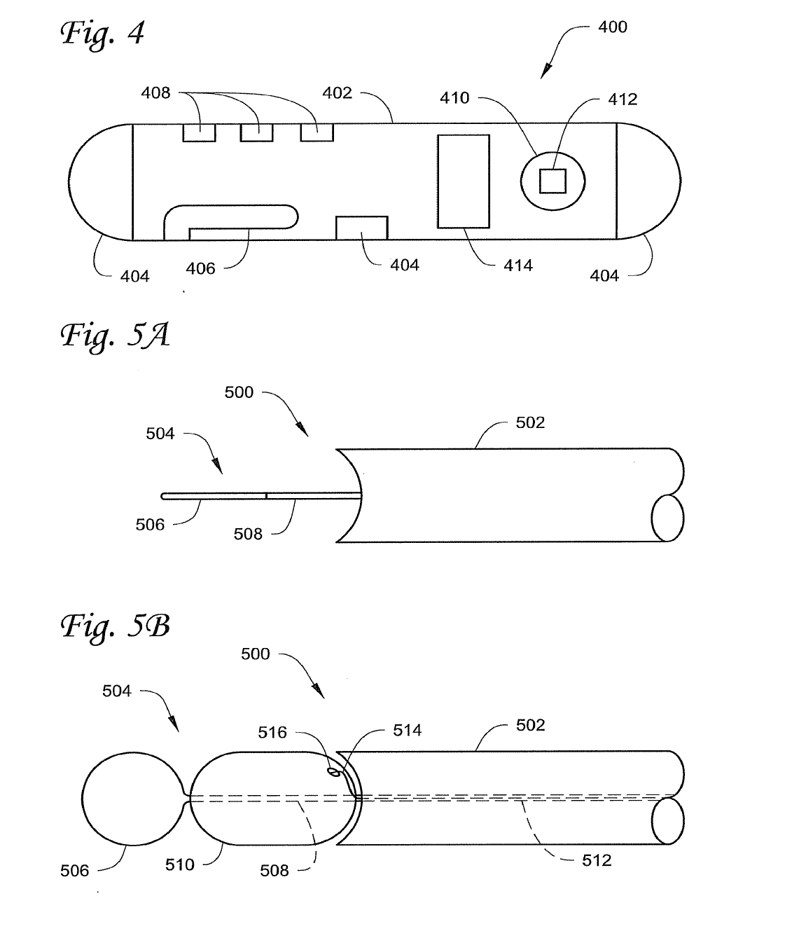

FIG. 4 shows another illustrative embodiment of a diagnostic capsule 400 as described herein. The diagnostic capsule 400 includes a housing 402 and several optional components. Diagnostic capsule 400 includes three imaging devices 404 (e.g., cameras, etc.), two of the imaging devices 404 located at opposite ends of the housing and one imaging device 404 along the length of the housing. The separation between the imaging devices may be useful in providing enhanced imaging of tissues, surfaces, etc. as described in connection with the embodiment of FIG. 3E. The diagnostic capsule 400 also includes a specimen collection reservoir 406, three manometers 408 for measurement of pressure, and motor 414, a driver 410 and a tail fin 412 for transport of the diagnostic capsule 400. The oblong shape may allow for more additional functionality to be added to the diagnostic capsule 400. The housing 402 may be made of, e.g., silicon- or polymer-based construction which may provide improved flexibility and comfort.

FIG. 5A shows a diagnostic capsule delivery system 500 according to the present disclosure. The diagnostic capsule delivery system 500 includes an obturator 502, a balloon 504 (shown in FIG. 5A in a deflated state) having an inflatable portion 506 and a neck portion 508. FIG. 5B shows the diagnostic capsule delivery system 500 of FIG. 5A including a diagnostic capsule 510. The diagnostic capsule 510 includes a through-channel through which the neck portion 508 of the balloon 504 extends. The through-channel of the diagnostic capsule 510 received the balloon 504 in the deflated state, after which the balloon 504 was inflated. In the inflated state, the balloon 504 may be used to secure the diagnostic capsule 510 to the distal end of the obturator 502, by, for example, applying a pulling force on the neck portion 508 of the balloon 504 while applying a pushing force on the obturator 502. In FIG. 5B, the obturator 502 is shown with a lumen 512 through which a fluid may pass into the balloon and through which a retrieval article 514 may extend from an eyelet 500 on the diagnostic capsule 510. When the diagnostic capsule 510 reaches the bladder, the balloon may be deflated and the diagnostic capsule removed from the balloon 504 and the obturator 502. For example, a plunger may be inserted through the obturator 502 to push the diagnostic capsule 510 from the obturator 502 and the balloon 504.

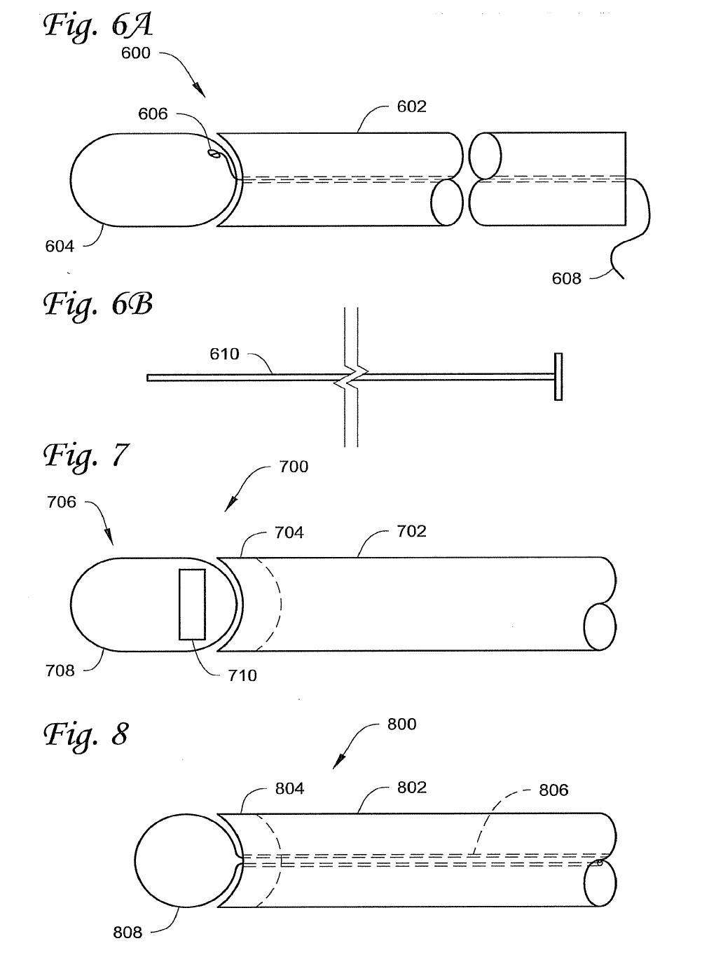

FIG. 6A shows a diagnostic capsule delivery system 600 according to the present disclosure. The diagnostic capsule delivery system includes an obturator 602, a diagnostic capsule 604 having an eyelet 606, and an optional retrieval article 608 extending from the eyelet 606 through the obturator 602 and extending out of the proximal end of the obturator 602. In one or more embodiments, obturator 602 may include a magnet in the distal end for attracting the diagnostic capsule 604. FIG. 6B shows a plunger 610 that may be used in the diagnostic capsule delivery system 600 of FIG. 6A to extend through the obturator 602 in order to disengage the diagnostic capsule 604 from the magnetic positioning element at the distal end of the obturator 602.

FIG. 7 shows a diagnostic capsule retrieval system 700 according to the present disclosure. The diagnostic capsule retrieval system 700 includes an obturator 702 having a magnetic distal end 704. The magnetic distal end 704 may be used to locate and secure to the magnetic distal end 704 a diagnostic capsule 706 having a housing 708 that includes an optional magnetic positioning element 710 that may be attracted to the magnetic distal end 704 of the obturator 702.

FIG. 8 shows a diagnostic capsule retrieval system 800 according to the present disclosure. The diagnostic capsule retrieval system 800 includes an obturator 802 having a magnetic distal end 804 and a lumen 806 passing through the obturator 802. A balloon 808 may extend through the lumen 806 and may be inflated such that the balloon inflates near the magnetic distal end 804. The diagnostic capsule retrieval system 800 may be advanced through a urinary tract until the balloon 808 and the magnetic distal end 804 extend into a bladder. The balloon 808 may then be deflated and may optionally be withdrawn. The magnetic distal end 804 of the obturator 802 may then be manipulated within the bladder until the magnetic distal end 804 attracts a magnetic positioning element of a diagnostic capsule and secures the diagnostic capsule thereto. The obturator 802 may then be withdrawn having the diagnostic capsule attached thereto.

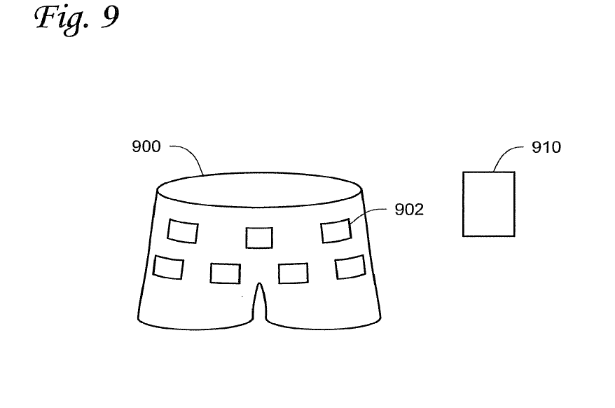

FIG. 9 is a block diagram of an optional undergarment 900 that may include a plurality of magnets 902 to assist in movement of a capsule as described herein. The system may also include a controller 910 that is operably connected to the magnets 902 to selectively activate the magnets to provide a selected combination of attractive and repulsive magnetic forces. In another alternative, the magnets 902 could also be attached directly to the skin of a patient using, e.g., adhesives, bands, etc.

The following provides further description of one or more embodiments of the present disclosure:

A diagnostic capsule as described herein may be used for bladder evaluations. A bladder installation device may include a loop incorporated into the end of a capsule-shaped medical device. This may permit a suture to be tied to the capsule and this may facilitate removal. A bladder installation device may have a rigid segment that may contact the capsule at the end that would be modified with the loop for the suture. When placing the capsule with the deployment device, traction may be applied on the suture and this may keep the capsule in contact with the rigid segment during placement. After the capsule is placed, the rigid segment would be removed after releasing the suture such that the capsule would remain within the bladder and the suture would be emanating from the urethra. The suture may be sufficiently long to permit placement and removal in the male and female.

In urology, a known procedure is the use of endoscopy to evaluate conditions in the urinary tract such as the monitoring of bladder cancer. This diagnosis may include that patients have periodic cystoscopic evaluations. This has been done using rigid or flexible instruments that are placed, for example, through the urinary channel and into the bladder, the ureter, or renal collecting system. Cystoscopy includes examination of the genitalia and can be associated with discomfort. It also may include a urologist performing the procedure.

The devices, systems, kits and methods described herein may provide an alternative to traditional endoscopy. Technicians may place capsules into the bladder or other areas of the urinary tract. The capsules may be placed using an introducer similar to having a urinary catheter placed. Once deployed, the capsule may record continuous images of the bladder lining. The capsule may be a freely mobile capsule endoscope adapted for urologic evaluation. For example, cameras may permit evaluation of the mucosa for pathologic changes. Sensors may also be incorporated that may monitor for cancer recurrence. Rather than have an uncomfortable examination, the patient may simply ambulate around the clinic. As the bladder fills the imaging and sensors may be active. Once the patient had a full bladder, then the patient may have a magnetic field generator moved systematically over the lower abdomen by a technician. The capsule may have a metal core (e.g., magnetic positioning element) that may move based on movement of the magnetic field generator. Accordingly, the entire bladder surface may be examined.

Diagnostic capsules, systems, kits, and methods as described herein may reduce physician involvement in the actual cystoscopic procedure. Examinations may be performed outside of the clinical setting. For instance, the examination could be performed in a nursing home, patient's residence, etc. The patient may need to have the device deployed by a health care professional, yet it is conceivable that a patient could be instructed as well to self-deploy the device. Equipment for recording after deployment would be available such that recording could be transmitted to the physician's office. This could eliminate the added expense of travel and inconvenience for the patient. A urologist may still be involved in the reading of the images, but increased efficiency may be realized.

Examinations of the urinary tract may be currently performed using rigid or flexible cystoscopes with instillation of saline to distend the bladder. This may cause patient discomfort. Other disadvantages of the current examinations may include institutional cost of cystoscopes and ureteroscopes, repair costs of cystoscopes, use of specialized cystoscopy suites, and use of direct urologist involvement during the entire procedure.

Capsules may be currently used for gastrointestinal applications clinically. The present disclosure may provide urological use of capsules, including improved deployment and retrieval techniques.

Use of wireless capsule endoscopy is described by Swain, “The future of wireless capsule endoscopy,” World J. Gastroenterol., 2008 Jul. 14, 14(26): 4142-4145.

Cystoscopy with a wireless diagnostic capsule (e.g., a capsule endoscope) may utilize RF (radio frequency) transmission or contact transmission. The diagnostic capsule may contain, for example, magnetic material and/or optical biopsy or cancer recognition technology.

A diagnostic capsule according to the present disclosure may be positioned within a bladder in order to, for example, capture and transmit images of, for example, bladder papilloma or a polyp. Such images may be transmitted such that the images may be view in real-time or may be stored for future viewing.

The one or more optional imaging devices described herein for use with the diagnostic capsules may include one or more video cameras and may be, for example, an ESO-type camera, which have been used in PillCam™ ESO devices to evaluate and diagnose diseases of the esophagus.

In one or more embodiments, a diagnostic capsule may have a retrieval device that is a wire. For example, a wire may be utilized to, for example, supply power to the diagnostic capsule from an external power source, which may reduce the need for batteries or other power sources in the diagnostic capsule. For example, a wire may be utilized as a conduit for images to be transmitted from the diagnostic capsule to an external storage device or viewing device. In one or more embodiments, a wire may provide power to the diagnostic capsule and have images transmitted therethrough.

In one or more embodiments, an optional magnetic positioning element may be provided inside the bladder and external to the diagnostic capsule housing. For example, a diagnostic capsule may have attached thereto an elongate tube having an inflatable balloon portion proximal to the diagnostic capsule and a source of a magnetic liquid (e.g., a magnetic or ferromagnetic slurry) distal to the diagnostic capsule. In one or more embodiments, the source of magnetic liquid may include a syringe or other device that is capable of transferring the magnetic liquid from the position distal to the diagnostic capsule through the elongate tube to cause the inflatable balloon portion of the tube to inflate and at least partially fill with at least a portion of the magnetic liquid. In one or more embodiments, the inflated balloon portion having magnetic liquid therein may be between the source and the diagnostic capsule, may be located on the opposite side of the diagnostic capsule relative to the attachment of the diagnostic capsule with the tube, or may be otherwise located proximal to the diagnostic capsule. The inflatable balloon portion of the tube having magnetic liquid therein may then be subject to magnetic forces from a magnetic field generator (e.g., a magnet external to the body in which the diagnostic capsule is located) to provide for manipulation of the diagnostic capsule within the body cavity.

A diagnostic capsule may be provided to, for example, a bladder using a diagnostic capsule delivery system that includes a guidewire. In one or more embodiments, a guidewire may be directed through, for example, the urinary channel and extending from inside a bladder to outside the body. A guidewire may be constructed of a wide variety of materials including metals and alloys. One having ordinary skill in the art would recognize these and other suitable materials for the guidewire. A distal end of the guidewire may be, for example, very flexible or floppy. A flexible dilator (e.g., a flexible plastic dilator) may be directed over the guidewire, the guidewire extending through a lumen in the flexible dilator. The flexible dilator may, for example, extend from inside the bladder to outside the body. The flexible dilator may also include a second lumen that may allow urine to drain from the bladder. A catheter may also be directed around the guidewire and may extend from the bladder to outside the body. With the catheter in place, the flexible dilator may be removed, leaving the guidewire extending through the catheter.

A diagnostic capsule as described herein that includes a through-channel may receive the guidewire within the through-channel. The diagnostic capsule may then be advanced along the guidewire, through the catheter and into the bladder with the use of an obturator (e.g., a pusher tube to push the diagnostic capsule through the catheter). In one or more embodiments, the catheter may further comprise a plug inserted in the proximal end of the catheter, wherein the plug includes a lumen through which a retrieval device may extend, a first port in order to receive a liquid (e.g., sterile water) to distend the bladder, and a second port having a tap capable of releasing liquid (e.g., water, urine, etc.) from the bladder.

Placement of a guidewire into the bladder can be accomplished using any one of a variety of techniques. One option would simply involve the placement of a Council tip catheter by the technician. A Council tip catheter has an opening at the distal tip that is designed to accommodate a guidewire. After placement of the Council tip catheter, a floppy guidewire would be placed. Using a push-pull movement, the Council tip catheter would be removed and the guidewire would be left inside the bladder. Now the capsule would be placed on the guidewire (like a bead on a string). The Council tip catheter or capsule introducer could then likewise be threaded over the wire. The wire could be held in place as the Council tip catheter or the introducer would push the capsule into the bladder. Return of urine would signal placement of the capsule into the bladder. At this point, the Council tip catheter/introducer and guidewire could be simultaneously removed.

In one or more embodiments, a diagnostic capsule delivery system may include a guidewire extending from a bladder to outside the body. A flexible catheter may be placed on the guidewire, wherein the catheter may provide some rigidity to the guidewire and wherein the catheter may be straight, curved, bent, or in some other configuration. A diagnostic capsule according to the present disclosure and having a through-channel receive the guidewire through the through-channel. Optionally, a cradle (e.g., a banana-skin cradle, which is soft and deformable) may be disposed around the diagnostic capsule before the guidewire is threaded through the through-channel of the diagnostic capsule. Other embodiments of the banana skin cradle could involve more rigid materials or basket wire type cradles. The cradle could potentially include as few as two flaps. This banana skin cradle would include a distal component of flaps that would hold the capsule in place and a proximal-overtube component as well. The last component would be an obturator that would fit into the overtube. Both the capsule and the obturator would have a center lumen for passing a guidewire. The first step would be to position the capsule into the flaps of the banana skin cradle. The second step would be to introduce the obturator into proximal overtube component and pass the obturator forward until it just contacts the capsule. The third step would be to feed the guidewire into the capsule (which is cradled by the banana cradle and stabilized by the obturator). During passage into the body cavity, the capsule would be kept in position by external forces applied by the tissues to the banana peel flaps. Once inside the body cavity, the guidewire and overtube would be stabilized. The obturator would now be advanced to disengage the capsule from the banana flaps. In this manner, the diagnostic capsule may be pushed along the guidewire and into the bladder with an obturator. The next step could include removal of the guidewire which would make the capsule fall into the bladder. The last step would be to remove the overtube.

In one or more embodiments, a diagnostic capsule delivery system may include a sheath that may be a peel-away sheath. A peel-away sheath may be elastic or may be capable of splitting from either end of the sheath, or from both ends of the sheath. For example, a peel-away sheath may be narrower at the distal end than the diagnostic capsule, wherein passage of the diagnostic capsule therethrough causes splitting or weakening of the sheath. A peel-away sheath may allow the sheath to be removed after the diagnostic capsule is in the bladder and may, for example, release pressure on the urethra. In one or more embodiments, the peel-away sheath may have a smaller diameter than the diagnostic capsule, but may be capable of allowing the diagnostic capsule to pass therethrough. In this embodiment, the capsule would be first backloaded into the peel away sheath. The peel away sheath may include three or more handles that would be present externally to the patient. Each handle would be attached to a flap with perforations that would comprise the peel away sheath. For a given peel away sheath, at least 50% of the flap would be folded back on itself before reaching the segment where the flaps are fused with the breakaway perforated zone. During installation, the force of entry would keep the capsule held in the peel-away sheath. The capsule that would be used for the peel away sheath in this embodiment would also have a central opening. Thus once the capsule/peel away sheath mechanism is in the bladder, return of urine would occur. At this point, the handles attached to the peel-away sheath would be pulled in a direction away from the patient and this would cause the perforations to split and the capsule would fall into the body cavity.

In one aspect, the present invention may provide diagnostic capsule comprising a housing; an eyelet formed in or otherwise attached to the housing, wherein the eyelet is capable of attachment to a retrieval article adapted to extend through a body lumen to permit retrieval of the diagnostic capsule, wherein the retrieval article optionally comprises a suture, a string, a wire, or a chain, and wherein the retrieval article has any suitable diameter and is optionally non-absorbable, antibacterial, coated, coiled, or braided; one or more optional magnetic positioning elements optionally within the housing, wherein the magnetic positioning element comprises a material attractive to a magnetic field generator; one or more optional lights within the housing; one or more optional imaging devices within the housing; one or more optional specimen collection reservoirs within the housing; one or more optional sensors within the housing, wherein the one or more optional sensors comprise one or more of the following optional sensors: a pressure manometer, an impedance sensor, a biomarker detection sensor, and a temperature sensor; one or more optional medical diagnostic devices within the housing, wherein the one or more medical diagnostic devices comprise one or more of the following medical diagnostic devices: an ultrasound device, a device for measurement of glucose metabolism, cancer recognition equipment, optical biopsy equipment; one or more optional transmitters within the housing, wherein the one or more transmitters can transmit data from the one or more optional transmitters to one or more optional receivers and wherein the data is optionally transmitted wirelessly; one or more optional motors within the housing; one or more optional transport mechanisms within the housing or connected to the housing; one or more optional through-channels extending from a distal end of the housing to a proximal end of the housing; one or more optional external magnets; and wherein the housing optionally comprises a silicone-based or polymer-based material; and wherein the housing is optionally oblong and optionally comprises a restrained linear configuration at least when located within a straight lumen and an unrestrained curvilinear configuration when located outside of the lumen.

In another aspect, the present invention provides a diagnostic capsule comprising a housing wherein the housing comprises a restrained linear configuration at least when located within a lumen of a delivery device and an unrestrained curvilinear configuration when located outside of the lumen; an optional eyelet formed in or otherwise attached to the housing, wherein the optional eyelet is capable of attachment to a retrieval article adapted to extend through a body lumen to permit retrieval of the diagnostic capsule, wherein the retrieval article optionally comprises a suture, a string, a wire, or a chain, and wherein the retrieval article has any suitable diameter and is optionally non-absorbable, antibacterial, coated, coiled, or braided; one or more optional magnetic positioning elements optionally within the housing, wherein the magnetic positioning element comprises a material attractive to a magnet or a material having a magnetic field; one or more optional lights within the housing; one or more optional imaging devices within the housing; one or more optional specimen collection reservoirs within the housing; one or more optional sensors within the housing, wherein the one or more optional sensors comprise one or more of the following optional sensors: a pressure manometer, an impedance sensor, a biomarker detection sensor, and a temperature sensor; one or more optional medical diagnostic devices within the housing, wherein the one or more medical diagnostic devices comprise one or more of the following medical diagnostic devices: an ultrasound device, a device for measurement of glucose metabolism, cancer recognition equipment, optical biopsy equipment; one or more optional transmitters within the housing, wherein the one or more transmitters can transmit data from the one or more optional transmitters to one or more optional receivers and wherein the data is optionally transmitted wirelessly; one or more optional motors within the housing; one or more optional transport mechanisms within the housing or connected to the housing; one or more optional external magnets; one or more optional through-channels extending from a distal end of the housing to a proximal end of the housing; and wherein the housing optionally comprises a silicone-based or polymer-based material.

In another aspect, the present invention provides a diagnostic capsule delivery system comprising a diagnostic capsule; an elongate obturator; a sheath having a proximal end, a distal end, and a first lumen extending therethrough and capable of receiving the obturator and the diagnostic capsule, wherein the sheath is optionally capable of extending from inside an animal's body to outside an animal's body, wherein inside the animal's body optionally comprises inside the animal's bladder, wherein the obturator is capable of extending from the proximal end of the sheath to the distal end of the sheath, wherein the obturator optionally comprises a second lumen for an optional passage of fluid, and wherein the passage of fluid is optionally into or out of an animal's bladder; an optional retrieval article attached to the eyelet, wherein the retrieval article is adapted to extend through a body lumen to permit retrieval of the diagnostic capsule, wherein the retrieval article optionally comprises a suture, a string, a wire, or a chain, and wherein the retrieval article has any suitable diameter and is optionally non-absorbable, antibacterial, coated, coiled, or braided;

wherein the sheath optionally further comprises a Y-shaped connector having a first arm, a second arm, and a connecting arm, wherein the first arm is optionally an irrigation port, wherein the second arm is optionally an obturator port.

In another aspect, the present invention provides a diagnostic capsule delivery system comprising: a diagnostic capsule; an elongate obturator; a sheath having a proximal end, a distal end, and a first lumen extending therethrough and capable of receiving the obturator and the diagnostic capsule, wherein the sheath is optionally capable of extending from inside an animal's body to outside an animal's body, wherein inside the animal's body optionally comprises inside the animal's bladder, wherein the obturator is capable of extending from the proximal end of the sheath to the distal end of the sheath, wherein the obturator optionally comprises a second lumen for an optional passage of fluid, and wherein the passage of fluid is optionally into or out of an animal's bladder; an optional retrieval article attached to the optional eyelet, wherein the retrieval article is adapted to extend through a body lumen to permit retrieval of the diagnostic capsule, wherein the retrieval article optionally comprises a suture, a string, a wire, or a chain, and wherein the retrieval article has any suitable diameter and is optionally non-absorbable, antibacterial, coated, coiled, or braided;

wherein the sheath optionally further comprises a Y-shaped connector having a first arm, a second arm, and a connecting arm, wherein the first arm is optionally an irrigation port, wherein the second arm is optionally an obturator port.

In another aspect, the present invention provides a diagnostic capsule delivery system comprising: a sheath having a proximal end, a distal end, and a first lumen extending therethrough; a balloon adapted for passing through the sheath while at least partially inflated, wherein the balloon has an inflatable end and a neck end; a diagnostic capsule further comprising a through-channel capable of receiving the neck end of the balloon, wherein the neck end extends through the through-channel of the diagnostic capsule; an elongate obturator; wherein the sheath is capable of receiving the obturator and the diagnostic capsule, wherein the sheath is optionally capable of extending from inside an animal's body to outside an animal's body, wherein inside the animal's body optionally comprises inside the animal's bladder, wherein the obturator is capable of extending from the proximal end of the sheath to the distal end of the sheath, wherein the obturator optionally comprises a second lumen for an optional passage of fluid, and wherein the passage of fluid is optionally into or out of an animal's bladder; wherein the passage of fluid is optionally into or out of the balloon; an optional retrieval article attached to the eyelet, wherein the retrieval article is adapted to extend through a body lumen to permit retrieval of the diagnostic capsule, wherein the retrieval article optionally comprises a suture, a string, a wire, or a chain, and wherein the retrieval article has any suitable diameter and is optionally non-absorbable, antibacterial, coated, coiled, or braided; wherein the sheath optionally further comprises a Y-shaped connector having a first arm, a second arm, and a connecting arm, wherein the first arm is optionally an irrigation port, wherein the second arm is optionally an obturator port.

As used herein and in the appended claims, the singular forms “a,” “and,” and “the” include plural references unless explicitly limited to the singular form or the context clearly dictates otherwise.

All references and publications cited herein are expressly incorporated herein by reference in their entirety into this disclosure. Illustrative embodiments of the diagnostic capsules, delivery/retrieval systems, kits and methods are discussed and reference has been made to possible variations within the scope of this invention. These and other variations and modifications will be apparent to those skilled in the art without departing from the scope of the invention, and it should be understood that this invention is not limited to the illustrative embodiments set forth herein. Accordingly, the invention is to be limited only by the claims and equivalents thereof.

1-29. (canceled)

30. A method of delivering a diagnostic capsule, the method comprising:
positioning a sheath in a body lumen such that a distal end of the sheath is located in a body cavity in communication with the body lumen and a proximal end of the sheath is located outside of a body; and
advancing a diagnostic capsule through the sheath towards the distal end until the diagnostic capsule exits the sheath and enters the body cavity.

31. A method according to claim 30, wherein advancing the diagnostic capsule comprises advancing the diagnostic capsule through the sheath by pushing the diagnostic capsule using an elongate obturator.

32. A method according to claim 31, wherein the elongate obturator comprises a distal end configured to cradle the diagnostic capsule as the diagnostic capsule is advanced through the sheath.

33. A method according to claim 32, wherein the distal end of the elongate obturator comprises a concave shape configured to receive a convex portion of the diagnostic capsule as the distal end of the elongate obturator and the diagnostic capsule are advanced through the sheath.

34. A method according to claim 31, wherein the method further comprises pulling a retrieval article towards the proximal end of the sheath while advancing the diagnostic capsule towards the distal end of the sheath, wherein the retrieval article is attached to the diagnostic capsule.

35. A method according to claim 31, wherein the diagnostic capsule comprises a through-channel and wherein a balloon extends through the through-channel such that the diagnostic capsule is located between an inflated portion of the balloon and the distal end of the elongate obturator, and wherein the method further comprises:
securing the diagnostic capsule to the distal end of the elongate obturator using the balloon; and
deflating the inflated portion of the balloon after the diagnostic capsule enters the body cavity; and
removing the balloon from the through-channel of the diagnostic capsule after deflating the inflated portion of the balloon.

36. A method according to claim 35, wherein securing the diagnostic capsule to the distal end of the elongate obturator comprises pulling the inflated portion of the balloon towards the distal end of the elongate obturator.

37. A method according to claim 35, wherein removing the balloon from the through-channel of the diagnostic capsule comprises pushing the diagnostic capsule from the balloon using a plunger inserted through the elongate obturator.

38. A method according to claim 30, the method further comprising removing the sheath from the body lumen after the diagnostic capsule enters the body cavity.

39. A method according to claim 38, wherein a retrieval article is attached to the diagnostic capsule, and wherein the method further comprises:
leaving the retrieval article in the body lumen after removing the sheath from the body lumen, wherein the retrieval article extends out of the body lumen outside of the body; and
removing the diagnostic capsule from the body cavity by pulling on the retrieval article.

40. A method according to claim 30, wherein a wire is attached to the diagnostic capsule, and wherein the method further comprises supplying power to the diagnostic capsule through the wire from an external power source.

41. A method according to claim 40, wherein the method further comprises transmitting images through the wire from the diagnostic capsule to an external device.

42. A method according to claim 30, wherein a wire is attached to the diagnostic capsule, and wherein the method further comprises transmitting images through the wire from the diagnostic capsule to an external device.

43. A method according to claim 30, wherein the method comprises removing the diagnostic capsule from the body cavity by:
advancing an elongate obturator such that a distal end of the elongate obturator is located in the body cavity;
magnetically attaching the diagnostic capsule to the distal end of the elongate obturator; and
removing the distal end of the elongate obturator and the diagnostic capsule magnetically attached to the distal end of the elongate obturator from the body cavity.

44. A method according to claim 43, wherein an inflated balloon is located near the distal end of the elongate obturator, and wherein the method comprises:
advancing the inflated balloon into the body cavity; and
deflating the inflated balloon after the inflated balloon enters the body cavity.

45. A method according to claim 30, wherein the body cavity comprises a bladder and wherein the body lumen comprises a urinary tract.

46. A method of delivering a diagnostic capsule, the method comprising:
positioning a sheath in a body lumen such that a distal end of the sheath is located in a body cavity in communication with the body lumen and a proximal end of the sheath is located outside of a body; and
advancing a diagnostic capsule through the sheath towards the distal end by pushing the diagnostic capsule using an elongate obturator until the diagnostic capsule exits the sheath and enters the body cavity;
removing the sheath from the body lumen after the diagnostic capsule enters the body cavity; and
removing the diagnostic capsule from the body cavity after removing the sheath from the body lumen, wherein a retrieval article is attached to the diagnostic capsule before the diagnostic capsule enters the body cavity, and wherein removing the diagnostic capsule comprises:
leaving the retrieval article in the body lumen after removing the sheath from the body lumen, wherein the retrieval article extends out of the body lumen outside of the body; and
removing the diagnostic capsule from the body cavity by pulling on the retrieval article.

47. A method according to claim 46, wherein the body cavity comprises a bladder and wherein the body lumen comprises a urinary tract.
